(19) United States
(12) Patent Application Publication
(10) Pub. No.: US 2002/0022015 A1
(43) Pub. Date: Feb. 21, 2002

(54) Administration system for seronegative of hepatitis C virus

(76) Inventor: Hiroaki Okushin, Himeji-shi (JP)

Correspondence Address: CROWELL & MORING, L.L.P., P.O. Box 14300, Washington, DC, 20044-4300, US

(21) Appl. No.: 09/950,774

(22) Filed: Sep. 13, 2001

Related U.S. Application Data
(63) Continuation of application No. 09/339,240, filed on Jun. 24, 1999, now abandoned.

(30) Foreign Application Priority Data
Jun. 24, 1998 (JP) JP10-176852

(51) Int. Cl. A61K 38/21

(52) U.S. Cl. 424/85.4

(57) ABSTRACT

The present invention provides an effective therapeutic agent for chronic hepatitis C, as well as a drug administration system for treating chronic hepatitis C by use of said therapeutic agent.

Specifically, the present invention provides a therapeutic agent for negation of hepatitis C virus (HCV)-RNA wherein the daily effective dosage of interferon (IFN) is administered once per day, or divided and administered in portions per day. The present invention also provides a drug administration system for administering said therapeutic agent, and a method for negation of HCV-RNA by said drug administration system, as well as a therapeutic method for chronic hepatitis C. The IFN is IFN-α or IFN-β.

### BACKGROUND OF THE INVENTION

1. Field of the Invention

The present invention relates to an effective therapeutic agent for chronic hepatitis C by negating hepatitis C virus(HCV)-RNA. Specifically, the present invention provides the effective therapeutic agent used for interferon (IFN) therapy, a drug administration system (drug administration unit), a method for negating HCV-RNA by said administration system, and further a therapeutic method for chronic hepatitis C by negation of HCV-RNA.

2. Description of the Prior Art

In general, hepatitis is understood to be an inflammation in the liver caused by viral infection in many cases, and hepatitis A caused by hepatitis A virus (HAV) and hepatitis B by hepatitis B virus (HBV) have been known for a long time. However, the presence of hepatitis non-A and non-B judged to be neither hepatitis A nor B came to be regarded as problematic, and thereafter, the presence of hepatitis C virus (HCV) as a major causative virus was revealed to be attributable to hepatitis non-A and non-B, and attention is now paid to chronic hepatitis C caused by HCV.

The characteristics of hepatitis C caused by HCV are high chronic degrees from acute hepatitis, as compared with other types of hepatitis. If chronic hepatitis C is unattended, the risk of progress through cirrhosis to hepatocellular carcinoma is high, so therapy effective against HCV should be conducted at an early stage.

Various IFN antiviral agents emerge as effective therapeutic agents for chronic hepatitis C. That is, it is known that IFN activates intracellular RNase thereby inhibiting the multiplication of RNA virus or DNA virus translated by reverse transcriptase. Because HCV as the causative virus for hepatitis C is also an RNA virus, various IFNs are used for the treatment of hepatitis C on the basis of its inhibitory action on viral multiplication.

IFN used so far for treatment of hepatitis C include IFN-α such as natural IFN-α, genetic recombinant IFN-α2a and IFN-α2b as well as natural IFN-β etc. These IFN are regarded as particularly effective for improvement of viral blood disease in chronic hepatitis C.

However, there is considerable difference in the clinical effect depending on the type of IFN. In particular, selection of IFN-α from several types of emerging IFN is very important but is not clarified much. Further, it is also true that there is a difference between IFN-α and IFN-β in clinical reactivity and viral therapeutic effect.

It is found that the therapeutic effect of IFN is dose-dependent, so the antiviral effect is higher when a higher dosage of IFN is administered. Accordingly, 3- to 10-million units are approved as the dosage of IFN-α administered in one portion for hepatitis C therapy, and the upper limit of 10-million units is often used while paying attention to side effects. Further, in respect of IFN-β, 3- to 6-million units is approved as a dosage administered in one portion, and in this case as well as, the upper limit of 6-million units is often used. This is based on the idea that HCV is rendered effectively negative at an early stage by the antiviral action of IFN to eliminate the virus.

However, intramuscular administration (muscular injection) is inevitable for IFN-α therapy, and the continuance of IFN in the blood (level peak time, half-time etc.) is long, causing a high burden on the patient (poor appetite, general dullness, depression etc.), and therefore causes some cases for administration to be terminated.

On the other hand, IFN-β is to be intravenously administered (intravenous injection or intravenous drip infusion), and the continuance of the IFN in the blood is relatively short as compared with IFN-α, and therefore, the burden on the patient is relatively small and the side effects are also of slight degrees. For example, in view of a patient given IFN-β, slight fever or headache is observed after initiation of its administration, but relieved after a relatively short period.

The difference between IFN-α and IFN-β is a difference in the administration route rather than the continuance thereof in the blood. However, from the view that the exhibition of the antiviral effect of IFN is originally due to inhibition of viral growth by binding IFN to IFN-receptors on the cell surface to induce antiviral proteins such as 2′-5′-oligoadenylate (2,5-AS), protein kinase or 2′-phosphadiesterase in cells rather than due to the direct action of IFN, it is rather preferable to administer IFN at a level enough to be able to bind to the receptors to actuate various signals and then to reduce the IFN level rapidly in order to prevent the down-regulation of the receptors.

Judging from this point, the intramuscular administration maintains high blood IFN levels, thus up-regulation of the receptors hardly occurs. Further the burden on the patient is high, and the occurrence of side effects is also considered to be high.

As described above, various pharmaceutical preparations of IFN is used for treatment of chronic hepatitis C, but under the present circumstances, there is no established therapeutic method which can demonstrate the working mechanism of the antiviral action of IFN while relieving the burden on the patient.

### SUMMARY OF THE INVENTION

In view of these circumstances, a fundamental problem of the present invention is to provide a more effective means of treating chronic hepatitis C by IFN.

To solve this problem, the present inventor focused his attention on the following points in treatment of chronic hepatitis C by IFN.

As the first point, the present inventor focused his attention on the feature that the antiviral action of IFN particularly induces antiviral proteins such as 2,5-AS etc. via receptors on the surface of infected cells. In such cases, rapid reduction in IFN levels after administration is rather preferable for preventing down regulation of IFN receptors, thus it can be said that intravenous administration is preferable as an original administration route for IFN.

As the second point, the present inventor focused on the fact that there is no clear correlation between the normalization of GPT and the cure rate of viral therapy against HCV although the therapeutic effect on chronic hepatitis C by administration of IFN makes use of the normalization of GPT (due to the action of depression of transamidase) as an indication of hepatic functions. For example, the comparison made for effectiveness of various IFN on chronic hepatitis C based on Japanese therapeutic results, the degree of normalization of GPT after termination of various kind of IFN administration is relatively high, about 52 to 67%. Nevertheless, the cure rate of viral therapy in this case is as low as 20 to 40% on average.

Judging from the fact that hepatitis C is originally an infection caused by HCV, the change of GPT should not be used as an indication of the antiviral effect of IFN. Instead, the negation of HCV-RNA at an early stage after administration of IFN administration should be used as an indication of the antiviral effect.

As the third point, the present inventor focused his attention on the fact that chronic hepatitis C is a hepatic inflammation caused mainly by hepatitis C virus. Accordingly, hepatitis C itself should be regarded as a viral infection, and the treatment of hepatitis C should be regarded to be similar to the treatment of general microbial infections or viral infections.

That is, the basic treatment of conventional inflammations is to eliminate microorganisms by administering a bactericide such as antibiotics and transferring the drug at high concentration to tissues of the infection site. Because hepatitis C is also an infection, fine transfer of IFN as an antiviral agent to the target organ (hepatic tissues) infected with HCV would be a basic therapeutic method.

Mura et al. measured and examined IFN levels in the blood and in major tissues with time passage in order to compare the difference between the intramuscular and intravenous administration of IFN-α (FPI-31) into male rats. As a result, they reported that IFN levels in hepatic tissues can be observed upon intravenous administration but cannot be detected upon intramuscular administration (Yakuri & Chiryou, 21:185-200, 1993).

This report indicates that the transfer of IFN to the liver as tissues infected with the virus is indefinite in the case of intramuscular administration of IFN. So a relatively high dosage of IFN should be administered to compensate for indefinite transfer, thus causing an increasing burden on the patient and simultaneously increasing the risk of side effects.

Accordingly, when hepatitis C is regarded as a viral infection, it is necessary to transfer IFN smoothly to the target organ infected with the virus, and therefore, it can be said that it is more preferable to select IFN capable of intravenous administration. In this case, IFN capable of intravenous administration at present is only IFN-β, but IFN-α etc. can be administered as long as its intravenous administration is made feasible.

On the basis of these viewpoints, a first choice among IFN in treating chronic hepatitis C is most preferably IFN-β. This is because IFN-β is the only IFN capable of intravenous administration and further the burden on the patient, upon administration thereof, is much less compared to other IFN.

From the foregoing, the present inventor judged that IFN-β among various IFN promises well for a more effective therapeutic method for treatment of chronic hepatitis C. Further with the recognition that hepatitis C is an infection, the present inventor speculated that an additional means similar to that of conventional administration against infections can be used in administration of IFN-β in order to achieve more superior therapeutic results.

The conventional treatment of chronic hepatitis C with IFN-β involves administering its daily effective dosage (especially, the most effective dosage) once per day via intravenous injection and conducting this administration every day for 6 weeks. The idea that the dosage is divided in portions and administered several times per day did not exist. However, considering the high transfer of IFN-β via intravenous injection to hepatic tissues and the short half-life thereof in the blood, it would not be required to administer the daily effective dosage at once by intravenous administration. In particular, in consideration of the pharmacodynamics of IFN-β, the daily effective dosage can be divided and administered in portions per day, and the present inventor judged that the administration of IFN in portions per day may be rather reasonable as a therapeutic method against the infection.

Accordingly, the daily effective dosage of IFN-β is divided into several portions, for example 2 to 3 portions, and divisional administration was conducted. As a result, effective induction of an antiviral protein 2,5-AS was confirmed thereby the antiviral effect of IFN-β could be persistent, and the negation of HCV-RNA was confirmed at a relatively early stage. The negation of HCV-RNA as the cure rate of viral therapy is very high such that it was hardly predictable from the conventional administration method conducted once per day, and the present invention was completed on the basis of this completely new finding.

Accordingly, the present invention provides as a first embodiment a therapeutic agent for chronic hepatitis C wherein interferon (IFN) can be divided and administered in portions e.g. 2 to 4 portions per day and its total dosage is a daily effective dosage of IFN.

Further, the present invention provides as a second embodiment a drug administration system (drug administration unit) for making HCV-RNA negative by use of said therapeutic agent for treating chronic hepatitis C, wherein the daily effective dosage of IFN is divided and administered in portions per day.

As another embodiment, the present invention provides a method of making HCV-RNA effectively negative by administration of IFN using said therapeutic agent in the above-described administration system.

The negation of HCV-RNA by this administration in portions provided by the present invention, as well as the therapeutic agent used thereof, can make use of either IFN-α or IFN-β among which IFN-β is preferable. Thus the present invention particularly provides a drug administration system wherein the daily effective dosage of IFN-β is divided in portions and administered several times per day to make HCV-RNA negative, as well as a therapeutic agent used for said drug administration system.

The frequency of administering the daily effective dosage of the therapeutic agent in portions per day and the frequency of administration in the administration system using said therapeutic agent are not particularly limited. The frequency shall be selected as such that negation of HCV-RNA as the object can be reliably achieved, and this frequency is determined depending on the type of IFN and the condition of the patient. If IFN-β is used, the administration in 2 to 4 portions per day, particularly in 2 portions per day, is preferable.

Further, the daily effective dosage of IFN is not particularly limited either. The daily effective dosage can be selected within the range of dosage used in general for treatment of chronic hepatitis C, for example within the range of 3- to 10-million units for IFN-α and 3- to 6-million units for IFN-β.

Six-month administration is approved for the conventional treatment of chronic hepatitis C by administration of IFN. If no effect is observed by the administration thereof, means such as terminating the administration etc. are adopted.

By the administration system using the therapeutic drug of the invention, the negation of HCV-RNA is observed at a relatively early stage. Accordingly, the period of the administration of the present invention shall be the period only required to negate HCV-RNA, and the administration shall be conducted preferably every day for 1 to 4 weeks.

Accordingly, the preferable specific administration system of the present invention is a drug administration system for negation of HCV-RNA wherein the daily effective dosage of IFN-β (3- to 6-million units) is divided and administered in 2 or 3 portions per day, and said administration in portions per day is conducted for 1 to 6 weeks.

By the specific administration system of the present invention as described above, HCV-RNA as a causative virus for hepatitis C is rendered negative at an early stage.

Accordingly, the present invention provides as a further embodiment a therapeutic method against chronic hepatitis C wherein HCV-RNA is made negative by use of the drug administration system described above. More specifically, the present invention provides a method of treating chronic hepatitis C by making HCV-RNA negative in the drug administration system wherein the daily effective dosage of IFN-β is divided and administered in 2 or 3 portions per day and said administration is conducted for 1 to 6 weeks.

The specific drug administration system for IFN using the therapeutic agent provided by the present invention is a drug administration method never examined before. By performing administration in portions per day, antiviral protein such as 2,5-AS etc. can be efficiently induced in cells infected with HCV whereby HCV-RNA is negated at an early stage. Further, transaminase levels such as GPT levels as an indication of hepatic functions are also effectively normalized. Therefore, from another viewpoint, the present invention also provides a method to induce 2,5-AS efficiently by the specific administration system of IFN.

Considering the fact that the cure rates achieved by administration of IFN in one portion per day for chronic hepatitis C was about 20% on average, the drug administration system of the present invention can be said to be particularly superior. It can be even said that it is epoch-making in providing a new guideline for therapy of chronic hepatitis C.

As described above, the present invention also provides a method to efficiently induce a group of proteins (e.g. 2,5-AS) induced by the specific administration system of IFN using the therapeutic agent of the present invention. And it was confirmed that such induction of 2,5-AS is effective for making HCV-RNA negative by the administration system of the present invention wherein IFN-β is divided in portions and administered several times per day.

### DETAILED DESCRIPTION OF THE INVENTION

Hereinafter, the therapeutic drug of the present invention against chronic hepatitis B, the specific drug administration system of IFN thereof, and negation of HCV-RNA in said system using IFN-β as IFN are described in detail by reference to the Examples.

It should be noted that the basic administration system for any other kinds of IFN is the same as for IFN-β described below.

The daily effective dosage of IFN-β approved at present in the world ranges from 3- to 6-million units. Considering that hepatitis C is an infection caused by HCV, the maximum dosage of 6-million units per day, that is the more effective daily dosage in the present invention, is preferably used, and said dosage is divided and administered preferably in portions per day.

However, the dosage can naturally be selected within the range of 3- to 6-million units as the daily effective dosage, according to the 6-stage evaluation from − to 5+ as determined by the HCV-RNA semi-quantification method.

For example, 6-million units as the maximum of the daily effective dosage of IFN-β were divided into 2 portions, and each portion (i.e. 3-million units) was dissolved in 100 ml of 5% glucose solution and administered twice per day (total of 6-million units/day) via intravenous drip infusion for e.g. 20 to 30 minutes in the morning and evening (e.g. 9:00 and 19:00). As a result of such administration, HCV-RNA became negative at a relatively earlier stage than in conventional methods. In particular, it was revealed that when bDNA levels were 1.0 Meq/ml or less determined by the branched DNA probe method (bDNA—Daiichi Kagaku Yakuhin), there were many cases where HCV-RNA could be rendered negative even only 1 week after the administration was initiated, and the HCV-RNA was completely negative 2 weeks after the administration was initiated.

In this example, IFN-β was divided and administered in portions at 9:00 in the morning and at 19:00 in the evening. Nevertheless, this interval could be determined suitably depending on the divided dosage and the frequency of administration in consideration with the pharmacodynamics of IFN-β.

In this example, HCV virus was quantified not only by said bDNA probe method but also by a HCV-RNA semi-quantification method (Shionogi) using nested RT-PCR techniques as well as a HCV-RNA quantification method using PCR techniques (Amplicor HCV monitor method: qualitative, quantitative determination: Roche) etc.

As described above, in the condition where bDNA levels were 1.0 Meq/ml or less, negation of HCV-RNA was achieved almost completely. It was further confirmed that the system of dividing and administering IFN-β in portions per day, provided by the present invention, was effective for negation of HCV-RNA even in hardly remediable cases (with bDNA levels of 1.0 Meq/ml or more and genotype 1b). That is, by the administration in 2 portions per day, HCV-RNA in the hardly remediable cases was rendered negative 2 weeks after initiation in 15 cases out of all the 19 cases examined, and this negation (%) was 7.89% (15/19), thus indicating good viral effect.

The results are summarized in Table 1 below.

TABLE 1
Negation of HCV-RNA at an Early Stage by the
Administration System of the Present Invention in the Case of
Antiviral Amounts (bDNA 1.0 Meq/ml), 1B Type

Negation of HCV-RNA (%)

| | 1 week after | 2 weeks after |
|---|---|---|
| | initiation | initiation (nested |
| bDNA (Meq/ML) | (Amplicor) | PCR) |
| 1.0 to 5.0 | 2/10 | 8/10 |
| | (20.0) | (80.0) |
| 5.0 or more | 4/9 | 7/9 |
| | (44.4) | (77.8) |
| Total | 6/19 | 15/19 |
| | (31.6) | (78.9) |

The change with time in the negation of HCV-RNA, along with the number of platelets, GPT and 2′-5′-oligoadenylate (2,5-AS) was examined to confirm the specific therapeutic effect on hepatitis C, of the above specific administration system where IFN-β was divided and administered in portions per day e.g. in 2 portions per day.

As a result, the change of the number of platelets was decreased to a certain degree at Week 1 after administration was initiated. Then at Week 2 the change of the number of platelets seemed to remain the same, but at Week 3 and Week 4 there was a tendency that the reduction of the number of platelets continued. However, it was found that after the administration of IFN-β in 2 portions per day, the number of platelets was recovered by subsequent administration in 1 portion per day.

The change of GPT was increased at Weeks 2 and 3 after initiation of administration, but decreased at Week 4. The tendency of this increase in GPT level at Weeks 2 and 3, along with the negation of HCV-RNA at an early stage, could be attributable to the antiviral effect of IFN-β in hepatic tissues.

In fact, viewing the change in induction of 2,5-AS, it was revealed that 2,5-AS was effectively induced after the administration of IFN-β was initiated, and even thereafter, 2,5-AS remained at relatively high titer. The results are summarized in Table 2 below:

Unit: pmp l/dl

| | Levels Just Before | Week 2 After | Week 4 After |
|---|---|---|---|
| Case No. | Administration | Administration | Administration |
| 1 | 210 | 710 | 530 |
| 2 | 120 | 410 | 300 |
| 3 | 200 | 490 | 440 |
| 4 | 78 | 300 | 350 |
| 5 | 65 | 210 | 260 |
| 6 | 61 | 320 | 320 |
| 7 | 100 | 490 | 490 |
| 8 | 160 | 380 | 620 |
| 9 | 39 | 190 | 290 |
| 10 | 34 | 540 | 370 |

As the administration system of the invention where IFN is administered in portions per day, the above example indicated that the dosage of IFN-β was divided and administered in two 3-million portions per day (6-million units per day), and with this dosage given, the effective negation of HCV-RNA was confirmed about 2 weeks after the administration was initiated.

Nevertheless, the administration of a suitably reduced dosage (e.g. 2-million unit) in administration several times per day (e.g. two administrations per day) is preferred, when it is impossible to continue the administration of 3-million units/twice per day due to strong positive reaction to albuminuria or a reduction in blood platelets.

Judging from the foregoing and in consideration of the reduction in the number of platelets and albuminuria, the treatment of chronic hepatitis C in the administration system using the therapeutic agent provided by the present invention is conducted for 1 day to 6 weeks on average, preferably 2 to 6 weeks, while measuring the negation of HCV-RNA. In view of the fatigue of immune response, the most preferable period of the therapy to be 4 to 6 weeks based on the administration system of the present invention. By the administration system of the present invention, the negation of HCV-RNA can be achieved in most cases of chronic hepatitis C with HCV-RNA levels of up to 4+.

On the other hand, even in hardly remediable examples with HCV-RNA levels being 5+(with bDNA being 1.0 Meq/ml or more and genotype 1b), negation of HCV-RNA can be effectively achieved by the administration system where the daily effective dosage of IFN is divided and administrated in portions per day for 1 to 6 weeks. For the further persistent antiviral effect after administration was performed, the daily effective dosage of IFN is then administered once per day, and this administration cycle is repeated suitably, whereby a high cure rate of the viral therapy can be achieved even in the hardly remediable cases.

Accordingly, another embodiment of the present invention provides an administration system for negation of HCV-RNA wherein said daily effective dosage of IFN is divided and administered in portions per day, followed by the administration thereof once per day.

The above mentioned subsequent administration of the daily effective dosage once per day of IFN, after the daily effective dosage is administered several times per day in the drug administration system of the present invention, is performed every day for about 1 to 6 weeks, preferably 2 to 6 weeks. The IFN used therein may be IFN-α or IFN-β.

However, considering that IFN-β is preferably used in the initial administration in portions per day, it can be said that IFN-β is preferable for the subsequent administration in 1 portion per day.

Actual treatment of hardly remediable chronic hepatitis C is described in detail by reference to the following example of another therapy.

6-million units as the daily effective dosage of IFN-β was divided into two 3-million unit portions and each portion was dissolved in 100 ml of 5% glucose solution and administered twice every day for 20 to 30 minutes in the morning and evening (9:00 and 19:00) (total of 6-million units/day) for 4 weeks via intravenous drip infusion into a patient. The patient (male, 52 year old) had chronic hepatitis C where HCV-RNA was 5+ (with bDNA of 7.0 Meq/ml and genotype 1b), 108 copies/ml in the CRT-PCR method, and 620 Kcopies/ml in the Ampricor HCV monitor method.

Two weeks after administration of IFN-β was initiated, the negation of HDV-RNA was confirmed. On the other hand, the GPT level was decreased from the initial level (320 IU/L) after the administration was initiated. However, this level tended to increase gradually after Week 2, and a reduction in the number of platelets (at the initiation of administration: 12.5×10⁴/mm³; at Week 4: 3.0×10⁴/mm³) was observed.

After the administration in 2 portions per day for 4 weeks, 6-million units of IFN-β as the daily effective dosage was dissolved in 100 ml of 5% glucose solution and administered once a day via intravenous drip infusion in the morning (9:00) for 20 to 30 minutes, and this administration was continued for 2 weeks.

By continuing this administration in 1 portion per day, the increased GPT level was then decreased, and simultaneously not only the reduction in the number of platelets was stopped but also the platelets commenced to increase. HCV-RNA remained negative even during this administration period.

Further, the same administration in 2 portions per day as that described above was conducted for 1 week, and thereafter, the administration in 1 portion per day was conducted for 5 weeks (total administration dosage of IFN-β:504 MIU).

The result indicated that after total administration of the IFN-β, the negation of HCV-RNA was persistent, the GPT level was in a normal range (431 U/L or less), and recovery of the number of platelets was also observed (at the end of the administration: 12.5×10⁴ platelets/mm3). Further, as a result of diagnosis for 6 months thereafter, recurrence of hepatitis C was not observed, and complete recovery was confirmed.

In view of the results in the above examples, the present invention provides as a further specific embodiment a drug administration system wherein the daily effective dosage of IFN-β is divided and administered in portions per day, preferably in 2 portions, and said administration in portions per day is conducted every day for 1 day to 6 weeks. Subsequently the administration of the daily effective dosage of IFN-β in 1 portion per day is further conducted every day for 1 day to 4 weeks, and a cycle of the administration in portions per day and the administration in 1 portion per day is repeated if necessary. Further, the present invention provides a method for negation of HCV-RNA by said drug administration system as well as a therapeutic method for chronic hepatitis C by negation of HCV-RNA.

It is preferable to finish the repetition of the cycle of the administration in several portions per day and in 1 portion per day within 12 weeks in consideration of the burden on the patient. Therefore, the total dosage of IFN may be determined preferably in consideration of the viral therapy as negation of HCV-RNA.

As described above, the present invention provides a therapeutic agent of IFN against chronic hepatitis C as well as a specific administration system (administration unit) using said therapeutic agent. By this administration system, antiviral proteins such as 2,5-AS can be efficiently induced in infected cells whereby HCV-RNA is rendered negative at an early stage, resulting in treatment of chronic hepatitis C.

In considering the fact that the conventional therapy of chronic hepatitis C by administration of IFN has achieved an effectiveness of only 20%, indicating a limit to the IFN therapy, the therapeutic agent of the present invention is particularly superior and gives a new guideline for the therapy of chronic hepatitis C by IFN, thus making a great contribution to medicine.

The specific therapeutic agent provided by the present invention, as well as the negation of HCV-RNA by the administration system using said therapeutic agent, can be inevitably effective against chronic hepatitis C. Also it was surprisingly revealed that the administration of IFN, particularly IFN-β, by this administration system was effective against hepatocellular carcinoma or cases resembling hepatocellular carcinoma and further against cirrhosis.

If chronic hepatitis C is unattended, the degree of transition from cirrhosis to hepatocellular carcinoma is considerably high, and the transition to cirrhosis or hepatocellular carcinoma in this case is caused by HCV. From this viewpoint, even if the patient already had a cirrhosis, the negation of HCV-RNA constitutes an effective therapeutic method. Further, the cancer tissues are removed by surgery in the case of hepatocellular carcinoma, but the recurrence of the hepatocellular carcinoma after the surgery is considerably high. This is because even normal non-cancer tissues have already been infected with HCV causing transition to cancer after removal of the cancer tissues, thus the recurrence of the cancer cannot be inhibited without negation of the HCV.

The specific administration system of the present invention is effective for treatment of cirrhosis or hepatocellular carcinoma, particularly for negation of HCV-RNA after surgery of hepatocellular carcinoma, in considering that this system can achieve the negation of HCV-RNA at an early stage.

What we claim is:

1. A therapeutic agent for chronic hepatitis C wherein interferon (IFN) can be divided and administered in portions per day and its total dosage is a daily effective dosage of IFN.

2. A therapeutic agent according to claim 1 wherein the daily effective dosage of IFN is divided and administered in 2 to 4 portions per day.

3. A therapeutic agent according to claim 1 or 2 which is administered every day for 1 to 6 weeks.

4. A therapeutic agent according to any one of claims 1 to 3 wherein the IFN is IFN-α or IFN-β.

5. A drug administration system for negation of HCV-RNA wherein the daily effective dosage of IFN is administered once per day or divided and administered in several portions per day.

6. A drug administration system according to claim 5 wherein the administration in 1 portion per day or in several portions per day is conducted for 1 or more days.

7. A drug administration system according to claim 5 or 6 wherein the administration in portions per day is administered in 2 or 3 portions per day.

8. A drug administration system according to any one of claims 5 to 8 for negation of HCV-RNA wherein the daily effective dosage of IFN is divided and administered in several portions per day, and subsequently administration of 1 portion per day is further conducted.

9. A drug administration system according to any one of claims 5 to 8 for negation of HCV-RNA wherein the daily effective dosage of IFN is divided and administered in 2 or 3 portions per day for 1 day to 6 weeks, and subsequently the administration in 1 portion per day is conducted for 1 day to 6 weeks.

10. A drug administration system according to any one of claims 5 to 9 wherein the administration in several portions per day and in 1 portion per day are repeated suitably.

11. A drug administration system according to any one of claims 5 to 10 wherein the IFN is IFN-α or IFN-β.

12. A method for negation of HCV-RNA by the drug administration system described in any one of claims 5 to 11.

13. A therapeutic drug according to any one of claims 1 to 4 for use in the drug administration system described in claims 5 to 11.

14. A therapeutic method against chronic hepatitis C which comprises negation of HCV-RNA by the drug administration system described in any one of claims 5 to 11.
